(19) United States
(12) Patent Application Publication
(10) Pub. No.: US 2022/0378790 A1
(43) Pub. Date: Dec. 1, 2022

(54) PHARMACEUTICAL PREPARATION COMPRISING AN AMIDE DERIVATIVE INHIBITING THE GROWTH OF CANCER CELL AND A PHARMACEUTICAL PRODUCT CONTAINING THE SAME

(71) Applicant: Hanmi Pharm Co., Ltd., Gyeonggi-do (KR)

(72) Inventors: Young II Kim, Gyeonggi-do (KR); Taek Kwan Kwon, Gyeonggi-do (KR); Ho Taek Im, Gyeonggi-do (KR); Yong II Kim, Gyeonggi-do (KR)

(73) Assignee: Hanmi Pharm Co., Ltd., Gyeonggi-do (KR)

(21) Appl. No.: 17/771,129

(22) PCT Filed: Oct. 23, 2020
(86) PCT No.: PCT/KR2020/014577

§ 371 Date: Apr. 22, 2022

(30) Foreign Application Priority Data
Oct. 24, 2019 (KR) 10-2019-0132809
Oct. 22, 2020 (KR) 10-2020-0137829

(51) Int. Cl. A61K 31/517; A61K 47/38; A61K 47/26; A61K 47/20; A61K 9/16; A61K 9/20

(52) U.S. Cl. CPC A61K 31/517; A61K 47/38; A61K 47/26; A61K 47/20; A61K 9/16; A61K 9/20

(57) ABSTRACT

The present invention relates to a pharmaceutical preparation comprising a granule comprising a compound of Chemical Formula 1 or a pharmaceutically acceptable salt thereof, and a diluent. The pharmaceutical preparation has a high productivity of the preparation due to excellent tableting properties, friability, and mass uniformity. The pharmaceutical preparation has low generated amount of impurities and high stability.

### TECHNICAL FIELD

The present invention relates to a pharmaceutical preparation comprising an amide derivative inhibiting the growth of cancer cells and a pharmaceutical product containing the same. Specifically, the present invention relates to a pharmaceutical preparation comprising a granule comprising a compound of Chemical Formula 1 or a pharmaceutically acceptable salt thereof, and a diluent, and a pharmaceutical product containing the same.

This application claims the benefit of priority based on Korean Patent Application No. 10-2019-0132809 filed on Oct. 24, 2019, and Korean Patent Application No. 10-2020-0137829 filed on Oct. 22, 2020, and the entire contents disclosed in the references of these Korean patent applications are hereby incorporated in their entirety as a part of the present specification.

### BACKGROUND ART

Epidermal growth factor receptor (EGFR) is known to be present as four subtype receptors of EGFR/ErbB1, Her-2/ErbB2, Her-3/ErbB3, and Her-4/ErbB4, and is abnormally overexpressed in most solid cancer cells. In addition, it is known that activation of receptors by ligands activates cellular signaling systems to induce growth, differentiation, neovascularization, metastasis, and resistance expression of cancer cells (Wells A., Int J Biochem Cell Biol., 1999, 31, 637-643). Therefore, according to the prediction that the anticancer effect will be excellent if the signal transduction of cancer cells through the epithelial growth factor receptors is blocked, research to develop anticancer agents targeting the epithelial growth factor receptors is actively underway.

These anticancer agents targeting the epithelial growth factor receptors are classified into monoclonal antibody drugs targeting the extracellular region of the receptors and low-molecular drugs targeting intracellular tyrosine kinase. The monoclonal antibody drugs have the advantages of exhibiting little side effects and excellent efficacy by selective binding to epithelial growth factor receptors. However, these drugs have disadvantages that they are not only expensive but also must be used in the form of injections. In contrast, the low-molecular drugs targeting tyrosine kinase are relatively inexpensive, may be administered orally, and have excellent efficacy by selectively or simultaneously acting on subtypes of epithelial growth factor receptors (EGFR, Her-2, Her-3, and Her-4).

The low-molecular drugs targeting the epithelial growth factor receptors include Iressa® (ingredient name: gefitinib; AstraZeneca), Tarceva® (ingredient name: erlotinib; Roche), which are selective inhibitors of EGFR, and Tykerv® (ingredient name: lapatinib; GlaxoSmithKline), which is a dual inhibitor that simultaneously blocks EGFR and Her-2, and these are used as therapeutic agents for lung cancer and Her-2 positive advanced breast cancer, respectively and are undergoing clinical trials to expand indications for treatment of other solid cancers.

In this regard, Korean Patent Application Laying-Open No. 10-2008-0107294 discloses a compound of following Chemical Formula 1 having less side effects, while selectively and effectively inhibiting the growth of cancer cells and resistance to drugs caused by EGFR and mutations thereof:

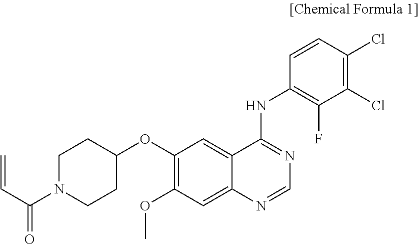 [SMILES: C=CC(=O)N1CCC(Oc2cc3c(Nc4ccc(Cl)c(Cl)c4F)ncnc3cc2OC)CC1]

However, with respect to a pharmaceutical composition comprising the compound, those skilled in the art have encountered problems of productivity and stability when preparing preparations such as tablets and capsules. Particularly, such compositions prepared by the way of conventional production steps typically suffer from a uniform purity, predictable stability, and shelf-life. Moreover, such compositions frequently suffer from significant capping and scuffing inconsistencies during the manufacturing phases, putting patients in a situation where they may receive suboptimal doses. Accordingly, to develop more suitable preparations and improve patient outcome, research on a pharmaceutical composition comprising the compound is continuously conducted.

### PRIOR ART REFERENCES

### Patent Documents

(Patent Literature 1) Korean Patent Application Laying-Open No. 10-2008-0107294

### DISCLOSURE OF INVENTION

### Technical Problem

With respect to a pharmaceutical preparation comprising Chemical Formula 1 above, it is intended to provide a pharmaceutical preparation having high productivity due to improved tableting properties, friability and mass uniformity, and high stability due to low generated amount of impurities even under severe conditions.

### Solution to Problem

The present invention addresses the shortcomings in the prior art.

According to a first aspect of the present invention,

The present invention provides a pharmaceutical preparation comprising a granule comprising a compound of following Chemical Formula 1 or a pharmaceutically acceptable salt thereof, and a diluent:

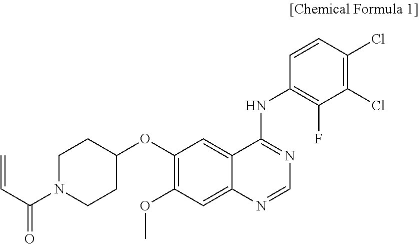 [SMILES: C=CC(=O)N1CCC(Oc2cc3c(Nc4ccc(Cl)c(Cl)c4F)ncnc3cc2OC)CC1]

In one embodiment of the present invention, the compound of Chemical Formula 1 or a pharmaceutically acceptable salt thereof is included in the pharmaceutical preparation in an amount of 2.0% or more and less than 20% by weight based on the total weight of the pharmaceutical preparation.

In one embodiment of the present invention, the diluent is included in the pharmaceutical preparation in an amount of 20% to 50% by weight based on the total weight of the pharmaceutical preparation.

In one embodiment of the present invention, the diluent is mannitol, microcrystalline cellulose, or a mixture thereof.

In one embodiment of the present invention, the diluent is a mixture of mannitol and microcrystalline cellulose in a weight ratio of 0.50:1 to 3.2:1.

In one embodiment of the present invention, the pharmaceutical preparation further comprises a glidant.

In one embodiment of the present invention, the glidant is selected from the group consisting of calcium stearate, magnesium stearate, sodium lauryl sulfate, zinc stearate, sodium benzoate, and mixtures thereof.

In one embodiment of the present invention, the glidant is included in the pharmaceutical preparation in an amount of 0.5% to 1.5% by weight based on the total weight of the pharmaceutical preparation.

According to a second aspect of the present invention,

the present invention provides a method of preparing the aforementioned pharmaceutical preparation, the method comprising the steps of: 1) mixing a compound of Chemical Formula 1 or a pharmaceutically acceptable salt thereof with a pharmaceutically acceptable additive and then performing granulation to prepare a granule; 2) mixing the granule with a pharmaceutically acceptable additive and then adding a diluent to prepare a mixed granule; and 3) formulating the mixed granule.

According to the third aspect of the present invention,

the present invention provides for methods of reducing impurities in a pharmaceutical preparation comprising the compound of Formula 1, by mixing granules containing the compound of Formula 1 with a pharmaceutically acceptable amounts of at least two diluents at suitable ratios and compressing such combinations into a tablet form wherein the amount of such impurities include Compounds of Formula 2 (also referred herein as Impurity IV) would be less than 1%, preferably less than 0.5%, more preferably less than 0.2% of the total weight of the preparation.

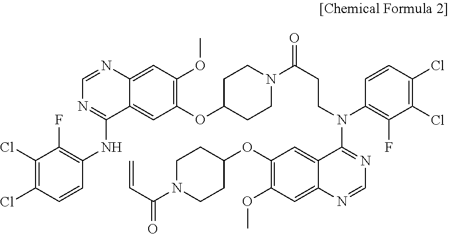 [SMILES: C=CC(=O)N1CCC(Oc2cc3c(N(CCC(=O)N4CCC(Oc5cc6c(Nc7ccc(Cl)c(Cl)c7F)ncnc6cc5OC)CC4)c4ccc(Cl)c(Cl)c4F)ncnc3cc2OC)CC1]

According to a fourth aspect of the present invention,

the present invention provides a pharmaceutical product in which the aforementioned pharmaceutical preparation is packaged in a packaging material.

In one embodiment of the present invention, the material of the packaging material is selected from the group consisting of glass, high density polyethylene (HDPE), polypropylene (PP), polyvinyl chloride (PVC), polyvinylidene chloride (PVDC), polychlorotrifluoroethylene (PCTFE), cycloolefin polymer (COP), cycloolefin copolymer (COC), polyolefin (PO), aluminum (Al), and combinations thereof, and the shape of the packaging material is selected from the group consisting of bottles, blisters, and pouches.

In one embodiment of the present invention, the packaging material comprises a moisture absorbent.

In one embodiment of the present invention, the moisture absorbent is calcium oxide or silica gel.

In one embodiment of the present invention, the silica gel is included in the packaging material in an amount of 2 to 5 g based on a 125 ml HDPE bottle.

According to the fifth aspect of the present invention,

the present invention provides a method for treating cancer in a subject in need thereof.

In one embodiment of the present invention, the subject has been determined to have one or more EGFR or HER2 activating mutations.

In one embodiment of the present invention, the method for treating the tumor includes administering a therapeutically effective amount of a pharmaceutical preparation comprising a granule comprising a compound of following Chemical Formula 1 or a pharmaceutically acceptable salt thereof, and a diluent, according to the present invention.

In one embodiment of the present invention, the cancer is selected from the group consisting of lung cancer, breast cancer, colorectal cancer, gastric cancer, brain cancer, cervical cancer, bladder cancer, bile duct cancer, ovarian cancer, pancreatic cancer, and testicular cancer.

In one embodiment of the present invention, the cancer is metastatic.

### Advantageous Effects of Invention

The pharmaceutical preparation, according to the present invention, is a pharmaceutical preparation comprising a compound of Chemical Formula 1, and has a high productivity of the preparation due to excellent tableting properties, friability, and mass uniformity by adding a granule comprising a compound of Chemical Formula 1 as an active ingredient and a specific diluent.

In addition, the present invention may provide a pharmaceutical product having low generated amount of impurities and high stability by specifying a metal salt glidant to be used in a pharmaceutical preparation and packaging the pharmaceutical preparation with a specific packaging material.

### BRIEF DESCRIPTION OF DRAWINGS

FIG. 1 is a graph showing the generated amount of impurity IV according to Experimental Example 6. The tablets of Example 1 and Comparative Examples 7 and 8 were packaged with Formpack® Dessiflex Blister (where to get: amcor) and left for 1, 2, and 4 weeks under 40° C./75% RH accelerated conditions, respectively, and then measured for impurity IV of Chemical Formula 2 by liquid chromatography.

FIG. 2 is a graph showing the generated amount of impurity IV according to Experimental Example 7. each of the tablets of Examples 6 to 9 and Comparative Example 9 above was packaged with Formpack® Dessiflex Blister (where to get: amcor), and the HM781-36B of Comparative Example 11 was packaged in an HDPE bottle, and then these were stored for 1, 2, and 4 weeks at a temperature of 60° C., which is a severe condition. The impurity IV of Chemical Formula 2 was measured for the samples stored for the above period according to the analysis conditions of Experimental Example 6.

FIG. 3 is a graph showing the generated amount of impurity IV according to Experimental Example 8. Each of the tablets according to Example 1 was packaged in either Al—Al blister, Al—PO+CaO—Al blister or HDPE bottle (5 different packaging with each fitted with a polypropylene cap including either 0.5, 2.0, 3.0, 4.0 or 5.0 g of silica gel and a polypropylene cap), wherein TEKNILID® 1207 (Tekniplex) was used for the Al—Al blister, Formpack® Dessiflex Blister (Amcor) was used for the Al—PO+CaO—Al blister, BTH-250 (Ewha Engineering) was used for the HDPE bottles, and the polypropylene cap (including silica gel) was also from Ewha Engineering with the proprietary names of MH-Cap (0.5 g), MH-Cap (2.0 g), MH-Cap (3.0 g), MH-Cap (4.0 g) and MH-Cap (5.0 g). The packaged products were left for 1, 2, and 4 weeks under 40° C./75% RH accelerated conditions, respectively, and then measured for impurity IV of Chemical Formula 2 according to the analysis conditions of Experimental Example 6.

### BEST MODE FOR CARRYING OUT THE INVENTION

Hereinafter, the present invention will be described in more detail.

A compound of following Chemical Formula 1 or a pharmaceutically acceptable salt thereof is very stable by itself, but a pharmaceutical preparation comprising the same exhibits a very unstable profile under severe conditions. Although the problem of instability was partially improved through the improvement of the packaging material, the fundamental stability of the pharmaceutical preparation was not improved.

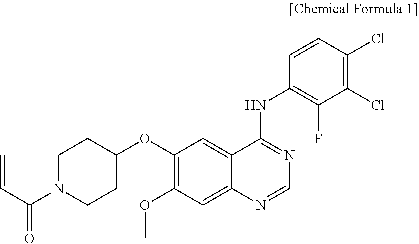 [SMILES: C=CC(=O)N1CCC(Oc2cc3c(Nc4ccc(Cl)c(Cl)c4F)ncnc3cc2OC)CC1]

Thus, with respect to the pharmaceutical preparation comprising Chemical Formula 1 above in the present invention, it is intended to provide a pharmaceutical preparation having high productivity due to improved tableting properties, friability and mass uniformity, and high stability due to low generated amount of impurities such as the impurity having the structure of Formula 2 even under severe conditions (60° C. for 1 month).

The present invention provides a pharmaceutical preparation comprising a granule comprising the compound of Chemical Formula 1 or a pharmaceutically acceptable salt thereof, and a diluent to be mixed with the granule.

The compound of Chemical Formula 1 (hereinafter referred to as code name HM781-36B) is a compound having less side effects, while selectively and effectively inhibiting the growth of cancer cells and resistance to drugs caused by EGFR and mutations thereof, as described in Korean Patent Application Laying-Open No. 10-2008-0107294.

The pharmaceutically acceptable salt of the compound of Chemical Formula 1 may be used in the form of a pharmaceutically acceptable salt derived from an inorganic or organic acid. Examples of the salts may be salts with inorganic acids such as hydrochloric acid, sulfuric acid, disulfuric acid, nitric acid, phosphoric acid, perchloric acid, bromic acid, and the like; or salts with organic acids such as formic acid, acetic acid, propionic acid, oxalic acid, succinic acid, benzoic acid, citric acid, maleic acid, malonic acid, malic acid, tartaric acid, gluconic acid, lactic acid, gestysic acid, fumaric acid, lactobionic acid, salicylic acid, phthalic acid, embonic acid, aspartic acid, glutamic acid, camsylic acid, besylic acid, or acetylsalicylic acid (aspirin). In addition, the pharmaceutically acceptable salt may be in the form of a metal salt obtained by reaction with an alkali metal such as calcium, sodium, magnesium, strontium, potassium, and the like.

The compound of Chemical Formula 1 or a pharmaceutically acceptable salt thereof may be included in an amount of 1.5% or more and less than 25% by weight, 2.0% or more and less than 20% by weight, 2.5% or more and less than 20% by weight, or 5% or more and less than 20% by weight preferably 3.5% to 15% by weight, and more preferably 5% to 8% by weight based on the total weight of the pharmaceutical preparation.

If the compound of Chemical Formula 1 or a pharmaceutically acceptable salt thereof is included in an amount of less than 2.0% by weight, tableting properties and dissolution rate are excellent, but stability is very poor to rapidly generate impurities, and if it is included in an amount of 20% by weight or more, the total content of the tablet decreases to reach a mass (less than 70 mg) in which tableting is impossible, and thus there is a problem that tableting is impossible.

In addition, the compound of Chemical Formula 1 or a pharmaceutically acceptable salt thereof may be included in an amount of 0.1 to 100 mg, preferably 0.5 to 50 mg.

The pharmaceutical preparation may be, for example, in the form of a powder, tablet, pill, capsule, liquid, suspension, emulsion, syrup, or granule, and preferably may be a tablet or capsule, but is not limited thereto.

The pharmaceutical preparation may further comprise a diluent, a binder, a disintegrant, and a glidant as a pharmaceutically acceptable additive. In some embodiments, the diluent may be a combination of at least two different diluents.

In an embodiment of the present invention, the pharmaceutical preparation may comprise the compound of Chemical Formula 1 or a pharmaceutically acceptable salt thereof prepared in the form of a granule. The granule may be prepared by mixing a compound of Chemical Formula 1 or a pharmaceutically acceptable salt thereof with a diluent, and then wet-granulating it in a binder solution in which a binder is dissolved in purified water.

The diluent may be one or more selected from the group consisting of mannitol, microcrystalline cellulose, lactose, and calcium phosphate, and preferably may be mannitol, microcrystalline cellulose or a mixture thereof. In addition, the diluent may be included in an amount of 50% to 99% by weight, preferably 60% to 95% by weight, and more preferably 70% to 90% by weight based on the total weight of the granule. In some embodiments, the diluent may be a combination of mannitol and microcrystalline cellulose.

The binder may be one or more selected from the group consisting of povidone, hydroxypropyl cellulose, hydroxypropyl methyl cellulose, polyvinyl alcohol, and carboxymethyl cellulose, and preferably may be povidone, but is not limited thereto. The binder may be included in the granule in an amount of 0.5% to 10% by weight, preferably 1% to 7% by weight, and more preferably 2% to 5% by weight based on the total weight of the granule.

The granule may be mixed with an additional diluent and then purified to prepare a pharmaceutical preparation. The diluent to be mixed with the granule is physically separated from the diluent used in the preparation of the granule and has different functionality, and thus it is separated from each other and used independently. The diluent to be mixed with the granule may be preferably mannitol, microcrystalline cellulose, or a mixture thereof, and more preferably may be a mixture of mannitol and microcrystalline cellulose. The mixture of mannitol and microcrystalline cellulose may be a mixture of mannitol and microcrystalline cellulose in a weight ratio of 0.25:2 to 4:1.5, 0.75:1.25 to 3.5:1.25, or 0.50:1 to 3.2:1, preferably 1:1 to 2:1. The diluent to be mixed with the granule may be included in an amount of 20% to 50% by weight, and preferably 30% to 40% by weight based on the total weight of the pharmaceutical preparation. The choice of the diluent to be mixed with the granule may actually affect the productivity of the pharmaceutical preparation. Specifically, by selecting the aforementioned diluent, tableting properties and friability of the tablet may be improved, and a tablet having a uniform mass may be obtained.

In at least one embodiment, the diluent that is mixed with the granule may comprise two types of diluents. In some embodiments, the first type of diluent is selected from the group consisting of a lactose, mannitol, calcium sulfate, sucrose, dextrose, sorbitol, maltitol, and starch, while the second diluent is a cellulose derivative such as microcrystalline cellulose, hydroxypropylmethyl cellulose, caboxymethylcellulose and the like.

In at least one embodiment, the first diluent is mannitol and the second diluent is microcrystalline cellulose, wherein the weight ratio of mannitol to microcrystalline cellulose is in the ranges of 0.25:2 to 4:1.5; or 0.75:1.25 to 3.5:1.25; or preferably 0.50:1 to 3.2:1.

In some embodiments, the granule may be mixed with a disintegrant together with the aforementioned diluent, and then purified to prepare a pharmaceutical preparation. The disintegrant may be one or more selected from the group consisting of crospovidone, croscarmellose sodium, and sodium starch glycolate, preferably crospovidone, but is not limited thereto. The disintegrant may be included in an amount of 1% to 10% by weight, preferably 3% to 7% by weight based on the total weight of the pharmaceutical preparation.

In some embodiments, a glidant may be added prior to purification into the pharmaceutical preparation. According to one embodiment of the present invention, the glidant may be a metal salt glidant. The glidant may be one or more selected from the group consisting of calcium stearate, magnesium stearate, sodium lauryl sulfate, zinc stearate, and sodium benzoate, and preferably may be magnesium stearate. The glidant may be included in an amount of 0.5% or more and less than 5% by weight, preferably less than 2% by weight, and more preferably 0.5% to 1.5% by weight based on the total weight of the pharmaceutical preparation. The compound of Chemical Formula 1 or a pharmaceutically acceptable salt thereof may have poor stability when a glidant in the form of a metal salt is used, but the stability of the pharmaceutical preparation may be improved by including the glidant in an amount of 0.5% or more and less than 5% by weight.

In some embodiment, the present invention is substantially free (less than 1% by weight) of any acidic additives such as acetic acid, adipic acid, citric acid, ascorbic acid, erythorbic acid, lactic acid, propionic acid, tartaric acid, fumaric acid, formic acid, oxalic acid, camsylic acid, malic acid, maleic acid, edisylic acid, palmitic acid, stearic acid or even silicon dioxide. In some embodiment, the pharmaceutical preparation of the present invention contains less than 0.25% by weight of any such acidic additives. In some embodiments, the pharmaceutical preparation is free of any acidic additive.

In addition, the pharmaceutical preparation may have an outer surface coated with one coating base selected from the group consisting of an immediate-release film forming agent, an enteric coating base, and a sustained-release coating base, in order to prevent direct contact of the pharmacologically active ingredient with human hands or skin during handling.

The immediate-release film forming agent may be one or more selected from the group consisting of hydroxypropyl cellulose, hydroxypropyl methyl cellulose, polyvinyl alcohol, and polyvinyl alcohol-polyethylene glycol graft polymer, the enteric coating base may be one or more selected from the group consisting of (meth)acrylic acid copolymer, hydroxypropyl methyl cellulose phthalate, and cellulose phthalate acetate, and the sustained-release coating base may be one or more selected from the group consisting of cellulose acetate, ethyl cellulose, and polyvinyl acetate, but are not limited thereto.

The coating base may be included in an amount of 1% to 10% by weight, preferably 2% to 5% by weight based on the total weight of the pharmaceutical preparation. In certain embodiment, the coating layer in about 0.5 to about 5% of the total weight of the formulation, wherein the coating layer has less than 18 weight percent of titanium dioxide and no more than 25 weight percent polyvinyl alcohol, and optionally no more than 25 weight percent of lactose or talc.

In some embodiment, the coating base may be a polyvinyl acetate substrate that consists of only polyvinyl alcohol of any molecular weight or contained in a copolymer, for example, Kollicoat® IR (BASF, N.J. USA) or as part of a polyvinyl alcohol based coating system such as the various film coating products available under the trade name Opadry® (Colorcon, Pa., USA), for example, Opadry II® 85F series, Opadry® II 89F series or Opadry® white.

The pharmaceutical preparation of the present invention comprises the compound of Chemical Formula 1 or a pharmaceutically acceptable salt thereof and a pharmaceutically acceptable additive, wherein the compound of Chemical Formula 1 or a pharmaceutically acceptable salt thereof may be included in an amount of 5% or more and less than 20% by weight based on the total weight of the pharmaceutical preparation. At least one surprising observation is the improved stability, particularly the stability of the pharmaceutical preparation by suppressing the generation of impurities under severe conditions, without affecting tableting properties and dissolution rate. In some embodiments, the severe condition may include storage under 40° C. and 75% RH accelerated conditions for a duration of 1, 2, 4 weeks or 2, 3, 4, 5, or 6 months. In certain embodiments, the storage condition may include storage under 30° C. and 55% RH for a duration of 6 to 12 months.

The stability in at least some embodiments of the present invention, is assessed based on more than 5% change in assay from initial value of the Compound of Formula 1 or failure to meet the accepted criteria for potency when used in a biological or immunological procedure for its intended use, any degradation products exceeding acceptable criteria such as existence of an impurity, or failure to meet physical attributes, functionality test such as color, phase separation, caking, hardness of the final preparation.

In some embodiments, the present pharmaceutical preparation is in the form of a tablet, having a hardness of 4 to 20 kp, or preferably 6 to 17 kp by using suitable equipment.

The present invention provides a method of preparing the aforementioned pharmaceutical preparation. The method comprises the steps of: 1) mixing a compound of Chemical Formula 1 or a pharmaceutically acceptable salt thereof with a pharmaceutically acceptable additive and then performing granulation to prepare a granule; 2) mixing the granule with a pharmaceutically acceptable additive and then adding a diluent to prepare a mixed granule; and 3) formulating the mixed granule. Each step of the preparing method is specified according to the contents of the aforementioned pharmaceutical preparation.

The present invention provides a pharmaceutical product in which the aforementioned pharmaceutical preparation is packaged in a packaging material. The packaging material is to protect the preparation from light, heat, moisture, and the like, and the material of the packaging material may be selected from the group consisting of glass, high density polyethylene (HDPE), polypropylene (PP), polyvinyl chloride (PVC), polyvinylidene chloride (PVDC), polychlorotrifluoroethylene (PCTFE), cycloolefin polymer (COP), cycloolefin copolymer (COC), polyolefin (PO), aluminum (Al), and combinations thereof. The packaging material may have the aforementioned material and may be prepared in a form selected from the group consisting of bottles, blisters, and pouches. According to an embodiment of the present invention, the bottle may be a bottle made of HDPE, and the blister may be made of an upper plate comprising one or two or more materials selected from the group consisting of PVC, PVDC, PCTFE, PP, PE, COP, COC, PO, Al, and combinations thereof, and a lower plate comprising an Al material. The upper plate and/or lower plate may have a single structure or a double or more structure.

Glass, HDPE, PP, PVC, PVDC, PCTFE, COP, COC, PO, and Al used in the present invention may be those commonly used in the packaging of pharmaceutical products in the pharmaceutical field. For example, the HDPE may have a weight average molecular weight of about 50,000 to 150,000, and a density of about 0.941 g/cm³ to 0.965 g/cm³. The PP may have a weight average molecular weight of about 200,000 to 600,000, and the PVC may have a molecular weight distribution (Mw/Mn) of about 1.7 to 2.0 and a density of about 1.16 g/cm³ to 1.35 g/cm³. The PVDC may have a density of about 0.65 g/cm³ to 1.72 g/cm³, the PCTFE may have a specific gravity of about 2.12, and the PO, COP and COC may have a density of about 1.02 g/cm³ or less.

The packaging material according to the present invention may comprise a moisture absorbent. The moisture absorbent has a function of increasing the stability of the pharmaceutical preparation by controlling the moisture inside the packaging material. The moisture absorbent may be used without limitation as long as it is generally used in the relevant technical field, and preferably calcium oxide or silica gel may be used in relation to the active ingredient of the present invention. The moisture absorbent may be mixed with the material of the packaging material and applied to the packaging material in various forms. According to an embodiment of the present invention, in the case of using silica gel as the moisture absorbent, the silica gel may be preferably included in an amount of 2 to 5 g, preferably 3 to 5 g in the packaging material. The content of the silica gel is a value set based on a packaging material for packaging a preparation comprising about 480 mg of the active ingredient (HM781-36B) or a HDPE bottle with a capacity of about 125 ml. When the content of the silica gel is less than 2 g, the stability of the pharmaceutical preparation inside the packaging material may be lowered due to the inability to properly control the moisture inside the packaging material, and when the content of the silica gel is more than 5 g, the dissolution rate of the pharmaceutical preparation may be lowered by affecting the moisture of the pharmaceutical preparation itself.

The present invention also provides a method for treating cancer in a subject in need thereof. In some embodiments, the method for treating the cancer includes administering a therapeutically effective amount of a pharmaceutical preparation comprising a granule comprising a compound of following Chemical Formula 1 or a pharmaceutically acceptable salt thereof, and a diluent, according to the present invention. In some embodiments, the pharmaceutical preparation is substantially free of impurity IV.

In some embodiments, the cancer is selected from the group consisting of lung cancer, breast cancer, colorectal cancer, gastric cancer, brain cancer, cervical cancer, bladder cancer, bile duct cancer, ovarian cancer, pancreatic cancer, and testicular cancer. In some embodiments, the cancer is metastatic.

In some embodiments, the subject has been determined to have one or more EGFR or HER2 activating mutations. In some embodiments, the subject has been determined to have one or more HER2 activating mutations at one or more locations selected from the group consisting of Furin-like extracelluar region, transmembrane, and kinase domain.

In some embodiments, the subject has been determined to have one or more HER2 activating mutations selected from S310F/Y, I655V, V659E, R678Q, V697L, T733I, L755X, I767M, D769H/N/Y, V773M, V777L/M, L786V, V842I, and L869R.

In some embodiments, the subject has been determined to have solid tumors with EGFR activating mutations, wherein the subject may or may not have NSCLC or highgrade glioma). In some embodiments, the EGFR activating mutations are located in extracellular and/or transmembrane, including for example, EGFRvIII, R108K, R222C, A289T, P596L, G598V. In some embodiments, the EGFR activating mutations are located in kinase domain, including for example, EGFRvIII, R108K, R222C, A289T, P596L, G598V. Exon 20 insertion, E709K, G719X, V742I, E746_A750del, S768I, V769M, V774M, R831C, R831H, L858R, L861Q, A864V. In some embodiments, the subject has not received chemotherapy, biologics, immunotherapy, HER2 targeted therapy, curative-intent radiotherapy for the treatment of the cancer.

Hereinafter, preferred examples will be provided to help to understand the present invention, but the following examples are provided not to limit the present invention but to facilitate the understanding of the present invention.

### EXAMPLES

### Example 1

Tablets comprising the compound of Chemical Formula 1 (hereinafter referred to as “HM781-36B”, manufactured by Dongwoo Syntech Co., Ltd.) as active ingredients according to the composition described in Table 1 below were prepared.

Specifically, HM781-36B and D-mannitol (manufactured by Roquette) were wet granulated using a high shear mixer, and the wet granulation sieves while distributing HM781-36B with D-mannitol using a No. 35 sieve (500 μm). Then, povidone dissolved in an appropriate amount of purified water (manufactured by BASF) was added thereto to prepare a granular portion. The granules obtained through wet granulation were sieved using a No. 20 sieve (850 μm), and then dried using a fluid bed dryer (Fluid Bed Granulator). The above processes were repeated until a result of about 0.5% or less was obtained by measuring the numerical value of loss of drying.

The granular portion prepared through the above processes was mixed with a mixture of mannitol and microcrystalline cellulose (manufactured by Mingtai Chemical), and crospovidone (manufactured by BASF), and then magnesium stearate (manufactured by Peter Greven, Netherland) was added thereto and finally mixed. The resulting final mixture was prepared into a tablet having a hardness of about 5 to 10 kp by a conventional method using a tableting machine (manufactured by Sejong).

### Examples 2 to 5

Tablets comprising HM781-36B as active ingredients according to the composition described in Table 1 below were prepared in the same manner as in Example 1.

TABLE 1

| Processes | | Raw material | Example 1 | Example 2 | Example 3 | Example 4 | Example 5 |
|---|---|---|---|---|---|---|---|
| Wet | Mixing | HM781-36B | 8 | 8 | 8 | 8 | 8 |
| granulation | | Mannitol | 50 | 50 | 50 | 50 | 50 |
| | Binding | Povidone | 2 | 2 | 2 | 2 | 2 |
| | solution | <Purified water> | <8> | <8> | <8> | <8> | <8> |

| | | | | | | |
|---|---|---|---|---|---|---|
| Postmixing | Mannitol | 17 | 20 | 22.5 | 17.5 | 17 |
| | Microcrystalline | 17 | 14 | 11.5 | 17 | 16.5 |
| | cellulose | | | | | |
| | Crospovidone | 5 | 5 | 5 | 5 | 5 |
| Glidant | Magnesium | 1 | 1 | 1 | 0.5 | 1.5 |
| | stearate | | | | | |

| | | | | | |
|---|---|---|---|---|---|
| Total mass | 100 | 100 | 100 | 100 | 100 |

* The unit of the numerical value is mg/tablet, and purified water is removed during the process.

### Comparative Examples 1 to 8

Tablets comprising HM781-36B as active ingredients according to the composition described in Table 2 below were prepared in the same manner as in Example 1. In Table 2 below, Pruv® sodium stearyl fumarate from JRS PHARMA, dibasic calcium phosphate from Lian yungang Debang Fine Chemical, and pregelatinized starch from Roquette were used.

TABLE 2

| | | Raw | Comparative | Comparative | Comparative | Comparative | Comparative | Comparative | Comparative | Comparative |
|---|---|---|---|---|---|---|---|---|---|---|
| Processes | | material | Example 1 | Example 2 | Example 3 | Example 4 | Example 5 | Example 6 | Example 7 | Example 8 |
| Wet | Mixing | HM781-36B | 8 | 8 | 8 | 8 | 8 | 8 | 8 | 8 |
| granu- | | Mannitol | 50 | 50 | 50 | 50 | 50 | 50 | 50 | 50 |
| lation | Binding | Povidone | 2 | 2 | 2 | 2 | 2 | 2 | 2 | 2 |
| | solution | <Purified | <8> | <8> | <8> | <8> | <8> | <8> | <8> | <8> |
| | | water> | | | | | | | | |

| | | | | | | | | | |
|---|---|---|---|---|---|---|---|---|---|
| Postmixing | Mannitol | 11 | 26 | — | — | — | — | 17 | 17 |
| | Microcrystal | 23 | 8 | 34 | — | — | — | 16 | 17 |
| | line | | | | | | | | |
| | cellulose | | | | | | | | |
| | Lactose | — | — | — | 34 | — | — | — | — |
| | Dibasic | — | — | — | — | 34 | — | — | — |
| | calcium | | | | | | | | |
| | phosphate | | | | | | | | |
| | Pregelatinized | — | — | — | — | — | 34 | — | — |
| | starch | | | | | | | | |
| | Crospovidone | 5 | 5 | 5 | 5 | 5 | 5 | 5 | 5 |
| Glidant | Magnesium | 1 | 1 | 1 | 1 | 1 | 1 | 2 | — |
| | stearate | | | | | | | | |
| | Sodium | — | — | — | — | — | — | — | 1 |
| | stearyl | | | | | | | | |
| | fumarate | | | | | | | | |

| | | | | | | | | |
|---|---|---|---|---|---|---|---|---|
| Total mass | 100 | 100 | 100 | 100 | 100 | 100 | 100 | 100 |

* The unit of the numerical value is mg/tablet, and purified water is removed during the process.

### Examples 6 to 9 and Comparative Examples 9 to 10

The tablets were prepared as in Example 1, and then the tablets were coated using Opadary® 03F180000, and coated tablets comprising HM781-36B or a pharmaceutically acceptable salt were finally prepared. The compositions of Examples 6 to 9 and Comparative Examples 9 and 10 are shown in Table 3 below.

TABLE 3

| | | | | | | | Comparative | Comparative |
|---|---|---|---|---|---|---|---|---|
| Processes | | Raw material | Example 6 | Example 7 | Example 8 | Example 9 | Example 9 | Example 10 |
| Wet | Mixing | HM781-36B | 4 | 6 | 8 | 16 | 2 | 16 |
| granulation | | D-Mannitol | 35.2 | 37.5 | 50 | 100 | 50 | 34 |
| | Binding | Povidone | 1.4 | 1.5 | 2 | 4 | 1.5 | 1 |
| | solution | <Purified water> | <5.6> | <6> | <8> | <16> | <6> | <4> |

| | | | | | | | |
|---|---|---|---|---|---|---|---|
| Postmixing | D-Mannitol | 12.5 | 12.8 | 17 | 34 | 23.5 | 12 |
| | Microcrystalline cellulose | 12.5 | 12.8 | 17 | 34 | 17 | 12 |
| | Crospovidone | 3.6 | 3.8 | 5 | 10 | 5 | 2.5 |
| Glidant | Magnesium stearate | 0.8 | 0.8 | 1 | 2 | 1 | 0.5 |
| Coating | Opadry 03F180000 | 2 | 2.3 | 3 | 6 | 3 | 2 |

| | | | | | | |
|---|---|---|---|---|---|---|
| Total mass | 72.0 | 77.5 | 103 | 206 | 103 | 80 |

* The unit of the numerical value is mg/tablet, and purified water is removed during the process. HM781-36B in Examples 6 to 9 and Comparative Examples 9 and 10 above was included in an amount of 5.6%, 7.7%, 7.8%, 7.8%, 1.9% and 20% by weight, respectively, based on the total weight of the pharmaceutical preparation.

### EXPERIMENTAL EXAMPLES

### Experimental Example 1: Evaluation of Tableting Properties of Examples 1 to 5 and Comparative Examples 1 to 6

For Examples 1 to 5 and Comparative Examples 1 to 6 above, the flowability (Hausner ratio calculated by the formula H=ρ_{T}/ρ_{B} where ρ_{B} is the freely settled bulk density(g/mL) of the powder, and ρ_{T} is the tapped bulk density(g/mL) of the powder) of the final granules before tableting as well as the properties of capping and sticking in 100 tablets following the tableting step were determined and the results are shown in Table 4 below.

The flowability of the final granules before tableting is an indicator of how well the tablet flows, and the better the flowability, the higher the flowability in the process, which may be confirmed from indicating easy productivity. This is generally a pharmaceutical index and evaluated using a value called Hausner ratio, and the closer it is to 1, the better flowability may be expressed.

The freely settled bulk density (g/mL) in the Hausner ratio was calculated by weighing about 10 g of the final granules, placing them in a 50 mL measuring cylinder, and measuring the volume, and the tapped bulk density (g/mL) was calculated by measuring the freely settled bulk density, tapping the measuring cylinder on the floor, and measuring the volume when the volume no longer decreased.

The properties of the tablets were tested by visually determining whether capping and sticking occurred in each tablet for 100 tablets after tableting each tablet using the final granules.

TABLE 4

| | Hausner | Capping | Sticking |
|---|---|---|---|
| | ratio | (number) | (number) |
| Example 1 | 1.15 | 0 | 0 |
| Example 2 | 1.23 | 0 | 0 |
| Example 3 | 1.22 | 0 | 0 |
| Example 4 | 1.14 | 0 | 0 |
| Example 5 | 1.15 | 0 | 0 |
| Comparative Example 1 | 1.24 | 0 | 0 |
| Comparative Example 2 | 1.17 | 0 | 0 |
| Comparative Example 3 | 1.38 | 0 | 0 |
| Comparative Example 4 | 1.35 | 2 | 0 |
| Comparative Example 5 | 1.28 | 21 | 25 |
| Comparative Example 6 | 1.40 | 12 | 27 |

According to Table 4 above, in the case of using a diluent of mannitol, microcrystalline cellulose, or a mixture thereof during postmixing (Examples 1 to 5 and Comparative Examples 1 to 3), no capping or sticking phenomenon occurred, but in the case of using other diluents (Comparative Examples 4 to 6), it could be confirmed that a capping or sticking phenomenon occurred. In particular, in the case of using a diluent of a dibasic calcium phosphate or pregelatinized starch (Comparative Examples 5 and 6), a capping or sticking phenomenon of about 10% to 30% occurred, thereby resulting in more undesirable results.

In addition, it was determined when the diluents of lactose, dibasic calcium phosphate or pregelatinized starch (Comparative Example 4 to 6) as well as microcrystalline cellulose (Comparative Examples 3) were used alone as a single component, unexpected loss in fluidity was observed in tablets prepared with a Hausner ratio of 1.26 or more.

### Experimental Example 2: Evaluation of Tableting Properties of Examples 6 to 9 and Comparative Examples 9 and 10

Housener ratio, capping, and sticking were confirmed in the same manner as in Experimental Example 1.

TABLE 5

| | Hausner | Capping | Sticking |
|---|---|---|---|
| | ratio | (number) | (number) |
| Example 6 | 1.18 | 0 | 0 |
| Example 7 | 1.19 | 0 | 0 |
| Example 8 | 1.15 | 0 | 0 |
| Example 9 | 1.20 | 0 | 0 |
| Comparative Example 9 | 1.23 | 0 | 0 |
| Comparative Example 10 | 1.24 | — | — |

From the results of Table 5 above, it could be confirmed that all of Examples 6 to 9 and Comparative Examples 9 and 10 were measured to have a Hausner ratio of less than 1.26, and thus had excellent flowability.

In addition, after tableting into tablets, the capping and sticking for the tablets of Examples 6 to 9 and Comparative Example 9 were not visually observed. However, in the case of Comparative Example 10, the total mass of the tablet was 80 mg, which is a weight of 40% compared to Example 9 comprising the same HM781-36B, and thus it was confirmed to be unsuitable for the minimum amount of final granules required to prepare a tablet. Therefore, Comparative Example 10 was a final mixture in which tableting was impossible, and thus the other evaluations except for the evaluation of granules were not performed.

From the results above, it was surprisingly observed that even if the content of HM781-36B included in the pharmaceutical preparation increased, the properties of the tablet were not affected to the extent that the flowability and tableting of the granules was acceptable.

### Experimental Example 3: Evaluation of Friability of Examples 1 to 5 and Comparative Examples 1 to 4

For 65 tablets according to Examples 1 to 5 and Comparative Examples 1 to 4 above, the friability was measured with a friability meter (TAR 200 manufactured by ERWEKA, condition: 25 rpm, 4 minutes) and the results are shown in Table 6 below.

TABLE 6

| | Mass before | Mass after | |
|---|---|---|---|
| | measurement | measurement | Friability |
| | (mg) | (mg) | (%) |
| Example 1 | 6521.0 | 6516.4 | 0.07 |
| Example 2 | 6518.1 | 6510.3 | 0.12 |
| Example 3 | 6511.4 | 6507.5 | 0.06 |
| Example 4 | 6553.2 | 6546.6 | 0.10 |
| Example 5 | 6526.9 | 6523.6 | 0.05 |
| Comparative Example 1 | 6541.1 | 6533.3 | 0.12 |
| Comparative Example 2 | 6527.4 | 6513.7 | 0.21 |
| Comparative Example 3 | 6614.8 | 6608.2 | 0.10 |
| Comparative Example 4 | 6435.8 | 6383.0 | 0.82 |

According to Table 6 above, all of the tablets according to Examples 1 to 5 and Comparative Examples 1 to 4 showed a friability of 1% or less, but in the case of using a diluent of mannitol, microcrystalline cellulose, or a mixture thereof when postmixed (Examples 1 to 5 and Comparative Examples 1 to 3), it could be confirmed to exhibit more excellent friability.

### Experimental Example 4: Mass Deviation Test of Examples 1 to 5 and Comparative Examples 1 to 4

For 10 tablets according to Examples 1 to 5 and Comparative Examples 1 to 4, the mass deviation was measured, and the results are shown in Table 7 below.

TABLE 7

| | Average | Mean mass | Mass deviation |
|---|---|---|---|
| | mass | deviation | of 5% or more |
| | (mg) | (%) | (number) |
| Example 1 | 99.94 | 1.08 | 0 |
| Example 2 | 100.21 | 1.63 | 0 |
| Example 3 | 99.68 | 1.42 | 0 |
| Example 4 | 100.41 | 1.34 | 0 |
| Example 5 | 99.99 | 1.46 | 0 |
| Comparative Example 1 | 98.17 | 4.23 | 3 |
| Comparative Example 2 | 100.54 | 4.56 | 2 |
| Comparative Example 3 | 99.83 | 4.72 | 3 |
| Comparative Example 4 | 97.12 | 5.21 | 5 |

According to Table 7 above, in the case of using a mixture of mannitol and microcrystalline cellulose in a weight ratio of 1:1 to 2:1 as a diluent (Examples 1 to 5), it could be confirmed that tablets having a uniform mass were able to be obtained.

### Experimental Example 5: Dissolution Evaluation of Examples 6 to 9 and Comparative Example 9

The tablets of Examples 6 to 9 and Comparative Example 9 above were evaluated for dissolution using the dissolution conditions and analysis methods below. The evaluation results are shown in Table 8 below.

<Dissolution Conditions>

Dissolution Solution: Take 2 tablets and test in 900 mL of buffer solution, pH 1.2.
buffer solution, pH 1.2:HCl 7.0 mL and water were dissolved in 2.0 g of NaCl to make 1000 mL.

Device: Apparatus 2 method (paddle method) among USP <711> Dissolution items

Dissolution Temperature: 37±0.5° C.

Rotational Speed: 50±2 rpm

<HPLC Analysis Conditions>

Detector: Ultraviolet absorption spectrophotometer (measurement wavelength: 254 nm)

Column: Inertsil ODS-2, 4.6×150 nm, 5 μm or equivalent column

Mobile Phase: Acetonitrile:phosphate buffer solution (pH 2.5)=40:60

(phosphate buffer solution, pH 2.5: Prepared by dissolving 7.0 g of NaClO₄ and 1.7 g of KH₂PO₄ in 1 L of purified water and adjusting the pH to 2.5 with phosphoric acid.)

Analysis Time: 10 minutes

Column Temperature: 30° C.

Flow Rate: 1.0 mL/min

Injection Volume: 50 μL

TABLE 8

| | Time(min) | | | | | | |
|---|---|---|---|---|---|---|---|
| | | 0 | 5 | 10 | 15 | 30 | 45 |
| Comparative | Average | — | 73.5 | 81.6 | 88.9 | 92.1 | 94.2 |
| Example 9 | Standard | — | 3.0 | 1.8 | 1.0 | 0.7 | 0.7 |
| | deviation (SD) | | | | | | |
| Example 6 | Average | — | 72.1 | 80.3 | 84.7 | 88.4 | 91.2 |
| | Standard | — | 0.3 | 1.4 | 1.6 | 1.9 | 2.2 |
| | deviation (SD) | | | | | | |
| Example 7 | Average | — | 71.7 | 76.8 | 86.5 | 91.5 | 93.7 |
| | Standard | — | 0.2 | 0.9 | 1.7 | 1.6 | 1.4 |
| | deviation (SD) | | | | | | |
| Example 8 | Average | — | 70.9 | 78.6 | 84.6 | 90.8 | 92.6 |
| | Standard | — | 1.2 | 0.9 | 2.2 | 2.0 | 3.6 |
| | deviation (SD) | | | | | | |
| Example 9 | Average | — | 72.6 | 79.4 | 84.6 | 89.9 | 93.5 |
| | Standard | — | 6.0 | 3.1 | 1.8 | 2.9 | 3.1 |
| | deviation (SD) | | | | | | |

From the results of Table 8 above, when the dissolution pattern of the tablets of Examples 6 to 9 and Comparative Example 9 above at pH 1.2 was observed, even if the content of HM781-36B of Examples 6 to 9 was higher than that of Comparative Example 9, it could confirm that the dissolution pattern and the final dissolution rate were not affected.

### Experimental Example 6: Stability Test for Packaged Tablets (Example 1 and Comparative Examples 7 and 8)

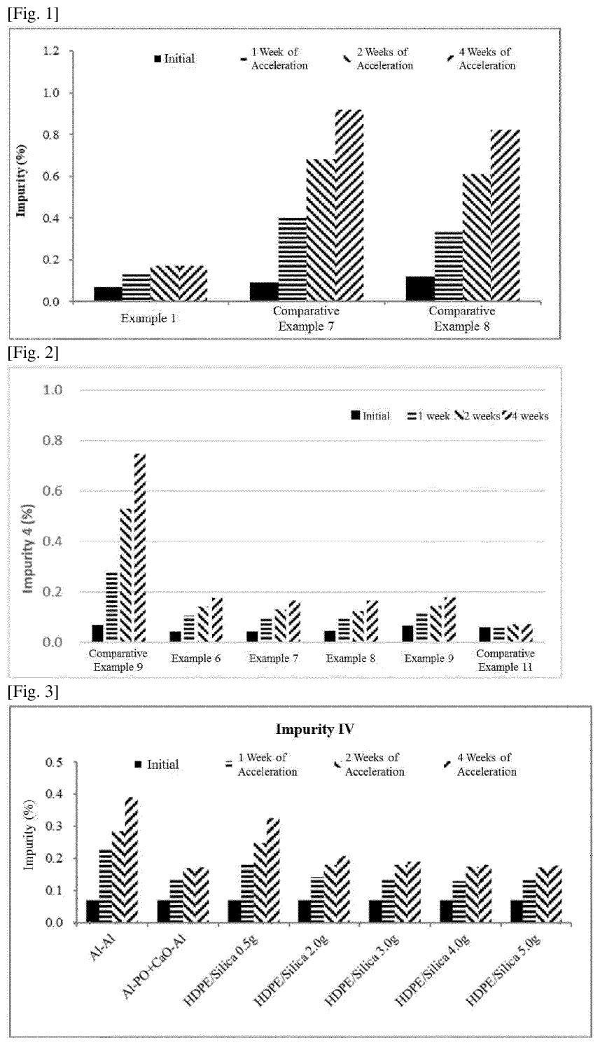

The tablets of Example 1 and Comparative Examples 7 and 8 were packaged with Formpack® Dessiflex Blister (where to get: amcor) and left for 1, 2, and 4 weeks under 40° C./75% RH accelerated conditions, respectively, and then measured for impurity IV of Chemical Formula 2 below by liquid chromatography (see analysis conditions below), and the results are shown in FIG. 1 and table 9.

<Analysis Conditions>

Detector: Ultraviolet absorption spectrophotometer (measurement wavelength: 254 nm)

Column: XTerra RP18, 4.6 mm×150 mm, 3.5 μm or equivalent column

Mobile phase: A—acetonitrile:phosphate buffer solution (pH 2.5)=40:60

B—acetonitrile:phosphate buffer solution (pH 2.5)=70:30

Column Temperature: 30° C.

Analysis Time: 45 minutes

Flow Rate: 1.0 mL/min

Injection Volume: 50 μL

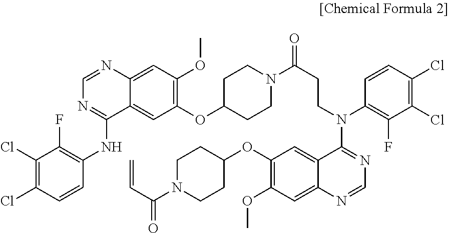 [SMILES: C=CC(=O)N1CCC(Oc2cc3c(N(CCC(=O)N4CCC(Oc5cc6c(Nc7ccc(Cl)c(Cl)c7F)ncnc6cc5OC)CC4)c4ccc(Cl)c(Cl)c4F)ncnc3cc2OC)CC1]

TABLE 9

| | Amount of Impurity IV(%) | | | |
|---|---|---|---|---|
| | | 1 Week of | 2 Weeks of | 4 Weeks of |
| | Initial | Accelation | Accelation | Accelation |
| Example 1 | 0.071 | 0.132 | 0.170 | 0.172 |
| Comparative Example 7 | 0.091 | 0.411 | 0.681 | 0.921 |
| Comparative Example 8 | 0.121 | 0.342 | 0.610 | 0.821 |

According to FIG. 1 and table 9, in the case of using a glidant of magnesium stearate in an amount of 2% by weight or more (Comparative Example 7), it was determined that the amount of impurity IV steadily increased in proportion to the time left to stand under accelerated conditions. In addition, in the case of using sodium stearyl fumarate, which is another metal salt glidant, in an amount of 1% by weight (Comparative Example 8), it was determined that the amount of impurity IV steadily increased in proportion to the time left to stand under accelerated conditions. Specifically, when left for 4 weeks under 40° C./75% RH accelerated condition, it was determined that the amount of impurity IV increased by 4 or more times in the tablet of Comparative Examples 7 and 8 compared to the tablets of Example 1, respectively.

From the results above, it was surprisingly observed that the type and content of the glidant comprised in the pharmaceutical formulation can affect the amount of impurity IV.

### Experimental Example 7: Stability Test for Packaged Tablets (Examples 6 to 9 and Comparative Examples 9 and 11)

The stability of the tablets of Examples 6 to 9 and Comparative Example 9 above was evaluated. In addition, the stability was evaluated using HM781-36B itself as Comparative Example 11.

Specifically, each of the tablets of Examples 6 to 9 and Comparative Example 9 above was packaged with Formpack® Dessiflex Blister (where to get: amcor), and the HM781-36B of Comparative Example 11 was packaged in an HDPE bottle, and then these were stored for 1, 2, and 4 weeks at a temperature of 60° C., which is a severe condition. The stability evaluation was performed for the samples stored for the above period according to the analysis conditions of Experimental Example 6. The stability evaluation is to measure impurity IV of Chemical Formula 2 below, and the results are shown in FIG. 2 and table 10.

TABLE 10

| | Amount of Impurity IV(%) | | | |
|---|---|---|---|---|
| | | 1 Week of | 2 Weeks of | 4 Weeks of |
| | Initial | Accelation | Accelation | Accelation |
| Comparative Example 9 | 0.07 | 0.28 | 0.53 | 0.75 |
| Example 6 | 0.05 | 0.11 | 0.14 | 0.18 |
| Example 7 | 0.05 | 0.09 | 0.13 | 0.17 |
| Example 8 | 0.05 | 0.09 | 0.13 | 0.17 |
| Example 9 | 0.07 | 0.11 | 0.15 | 0.18 |
| Comparative Example 11 | 0.06 | 0.06 | 0.07 | 0.07 |

According to FIG. 2 and table 10, since Comparative Example 11 comprises only HM781-36B, it showed very stable results for 4 weeks under severe conditions.

However, it was determined that the tablets of Examples 6 to 9 and Comparative Example 9, which were prepared by mixing the HM781-36B with a pharmaceutically acceptable additive, generated the impurity of Chemical Formula 2 above.

Specifically, Comparative Example 9 includes the HM781-36B in an amount of less than 2.0% by weight based on the total weight of the pharmaceutical preparation, and thus it was determined that the generated amount of the impurity of Chemical Formula 2 above significantly increased as time passed even when packaged in a stable packaging material. That is, even though the stability slightly increased from the packaging material, the results that did not improve the stability of the HM781-36B itself were showed.

However, Examples 6 to 9, which include the HM781-36B in an amount of 5% or more and less than 20% by weight based on the total weight of the pharmaceutical preparation, showed that the generated amount of the impurity of Chemical Formula 2 above did not increase significantly.

Specifically, when left for 4 weeks under 60° C. severe condition, it was determined that the amount of impurity IV increased by 3.5 or more times in the tablet of Comparative Example 9 compared to the tablets of Examples 6 to 9, respectively.

From the results above, it was surprisingly observed that the content of HM781-36B comprised in the pharmaceutical formulation can affect the amount of impurity IV.

### Experimental Example 8: Stability Test for Each Packaging Material of Tablet According to Example 1

Each of the tablets according to Example 1 was packaged in either Al—Al blister, AlPO+CaO—Al blister or HDPE bottle (5 different packaging with each fitted with a polypropylene cap including either 0.5, 2.0, 3.0, 4.0 or 5.0 g of silica gel and a polypropylene cap), wherein TEKNILID® 1207 (Tekniplex) was used for the Al—Al blister, Formpack® Dessiflex Blister (Amcor) was used for the Al—PO+CaO—Al blister, BTH-250 (Ewha Engineering) was used for the HDPE bottles, and the polypropylene cap (including silica gel) was also from Ewha Engineering with the proprietary names of MH-Cap (0.5 g), MH-Cap (2.0 g), MH-Cap (3.0 g), MH-Cap (4.0 g) and MH-Cap (5.0 g). The packaged products were left for 1, 2, and 4 weeks under 40° C./75% RH accelerated conditions, respectively, and then measured for impurity IV of Chemical Formula 2 above by liquid chromatography (see analysis conditions in Experimental Example 6), and the results are shown in FIG. 3 and table 11.

TABLE 11

| | Amount of Impurity IV(%) | | | |
|---|---|---|---|---|
| | | 1 Week of | 2 Weeks of | 4 Weeks of |
| | Initial | Accelation | Accelation | Accelation |
| Al—Al | 0.071 | 0.229 | 0.285 | 0.391 |
| Al—PO + CaO—Al | 0.071 | 0.132 | 0.170 | 0.172 |
| HDPE/Silica 0.5 g | 0.071 | 0.187 | 0.248 | 0.325 |
| HDPE/Silica 2.0 g | 0.071 | 0.142 | 0.181 | 0.207 |
| HDPE/Silica 3.0 g | 0.071 | 0.138 | 0.180 | 0.191 |
| HDPE/Silica 4.0 g | 0.071 | 0.131 | 0.176 | 0.181 |
| HDPE/Silica 5.0 g | 0.071 | 0.135 | 0.172 | 0.177 |

According to FIG. 3 and table 11, in the case of using an Al—PO+CaO—Al blister including CaO, which is a moisture absorbent, rather than an Al—Al blister, as a packaging material, it could be confirmed that the increased amount of impurity IV in proportion to the time left to stand under accelerated conditions decreased. In particular, in the packaging material of the Al—PO+CaO—Al blister, the increased amount of impurity IV remarkably decreased after 2 weeks of acceleration. In addition, in the case of using the HDPE bottle, it could be confirmed that the increased amount of impurity IV in proportion to the time left to stand under accelerated conditions decreased as the amount of silica gel, which is a moisture absorbent, increased in the cap. In particular, in the packaging material of HDPE bottles using a cap including 2 g or more of silica gel, the increased amount of impurity IV over time remarkably decreased.

### Experimental Example 9: Dissolution Test after Leaving for 4 Weeks Under Accelerated Conditions

Under the dissolution conditions and analysis conditions of Experimental Example 5, the dissolution rates of the tablets left for 4 weeks under accelerated conditions according to Experimental Example 8 were measured, respectively, and the results are shown in Table 12.

TABLE 12

| | Time(min) | | | | | | | |
|---|---|---|---|---|---|---|---|---|
| | | 0 | 5 | 10 | 15 | 30 | 45 | 60 |
| Al—Al | Average | — | 72.13 | 81.35 | 86.72 | 89.23 | 90.12 | 90.63 |
| | Standard | — | 3.0 | 2.2 | 1.5 | 0.7 | 0.5 | 0.3 |
| | deviation | | | | | | | |
| | (SD) | | | | | | | |
| Al—PO + | Average | — | 76.76 | 82.28 | 84.87 | 87.15 | 87.58 | 88.09 |
| CaO—Al | Standard | — | 2.5 | 2.2 | 1.1 | 0.8 | 0.5 | 0.8 |
| | deviation | | | | | | | |
| | (SD) | | | | | | | |
| HDPE/Silica | Average | — | 70.05 | 82.38 | 85.34 | 87.83 | 88.30 | 88.63 |
| 0.5 g | Standard | — | 2.7 | 1.9 | 1.4 | 1.0 | 0.8 | 0.9 |
| | deviation | | | | | | | |
| | (SD) | | | | | | | |
| HDPE/Silica | Average | — | 72.84 | 82.34 | 85.71 | 86.96 | 87.99 | 87.97 |
| 2.0 g | Standard | — | 1.9 | 1.8 | 1.9 | 0.9 | 0.9 | 0.7 |
| | deviation | | | | | | | |
| | (SD) | | | | | | | |
| HDPE/Silica | Average | — | 74.47 | 79.97 | 82.72 | 85.89 | 88.66 | 90.24 |
| 3.0 g | Standard | — | 3.4 | 2.7 | 2.1 | 1.8 | 1.5 | 0.4 |
| | deviation | | | | | | | |
| | (SD) | | | | | | | |
| HDPE/Silica | Average | — | 68.32 | 80.43 | 83.68 | 85.51 | 87.29 | 87.04 |
| 4.0 g | Standard | — | 4.1 | 2.5 | 1.5 | 0.8 | 1.3 | 1.1 |
| | deviation | | | | | | | |
| | (SD) | | | | | | | |
| HDPE/Silica | Average | — | 50.13 | 67.20 | 77.23 | 82.04 | 85.67 | 86.13 |
| 5.0 g | Standard | — | 8.0 | 3.7 | 2.8 | 1.7 | 1.4 | 1.3 |
| | deviation | | | | | | | |
| | (SD) | | | | | | | |

According to Table 12 above, in the case of using the HDPE packaging material using a cap including 5.0 g of silica gel, it could be confirmed that the tablets left for 4 weeks under accelerated conditions had a slow initial disintegration and a lower dissolution rate. However, after a period of about 60 minutes, the difference in the dissolution rate of each tablet was not large.

It should be appreciated that all of the simple modifications and variations of the present invention are within the scope of the present invention, and the specific scope of the present invention to be protected will be defined by the appended claims.

1. A pharmaceutical preparation comprising a granule comprising a compound of Chemical Formula 1 or a pharmaceutically acceptable salt thereof and a diluent to be mixed with the granule:

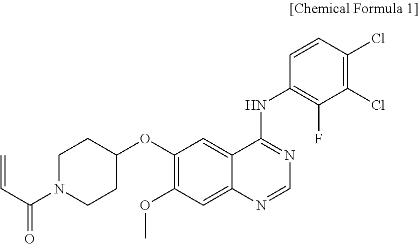 [SMILES: C=CC(=O)N1CCC(Oc2cc3c(Nc4ccc(Cl)c(Cl)c4F)ncnc3cc2OC)CC1]

2. The pharmaceutical preparation according to claim 1, wherein the compound of Chemical Formula 1 or the pharmaceutically acceptable salt thereof is included in the pharmaceutical preparation in an amount of 2.0% or more and less than 20% by weight based on the total weight of the pharmaceutical preparation.

3. The pharmaceutical preparation according to claim 1, wherein the diluent is included in the pharmaceutical preparation in an amount of 20% to 50% by weight based on the total weight of the pharmaceutical preparation.

4. The pharmaceutical preparation according to claim 1, wherein the diluent is mannitol, microcrystalline cellulose, or a mixture thereof.

5. The pharmaceutical preparation according to claim 4, wherein the diluent is a mixture of mannitol and microcrystalline cellulose in a weight ratio of 0.50:1 to 3.2:1.

6. The pharmaceutical preparation according to claim 1, wherein the pharmaceutical preparation further comprises a glidant.

7. The pharmaceutical preparation according to claim 6, wherein the glidant is selected from the group consisting of calcium stearate, magnesium stearate, sodium lauryl sulfate, zinc stearate, sodium benzoate, and mixtures thereof.

8. The pharmaceutical preparation according to claim 6, wherein the glidant is included in the pharmaceutical preparation in an amount of 0.5% to 1.5% by weight based on the total weight of the pharmaceutical preparation.

9. A method of preparing the pharmaceutical preparation according to claim 1, the method comprising the steps of:
1) mixing a compound of Chemical Formula 1 or a pharmaceutically acceptable salt thereof with a pharmaceutically acceptable additive and then performing granulation to prepare a granule;
2) mixing the granule with a pharmaceutically acceptable additive and then adding a diluent to prepare a mixed granule; and
3) formulating the mixed granules.

10. A pharmaceutical product in which the pharmaceutical preparation according to claim 1 is packaged in a packaging material.

11. The pharmaceutical product according to claim 10, wherein the material of the packaging material is selected from the group consisting of glass, high density polyethylene (HDPE), polypropylene (PP), polyvinyl chloride (PVC), polyvinylidene chloride (PVDC), polychlorotrifluoroethylene (PCTFE), cycloolefin polymer (COP), cycloolefin copolymer (COC), polyolefin (PO), aluminum (Al), and combinations thereof, and the shape of the packaging material is selected from the group consisting of bottles, blisters, and pouches.

12. The pharmaceutical product according to claim 10, wherein the packaging material comprises a moisture absorbent.

13. The pharmaceutical product according to claim 12, wherein the moisture absorbent is calcium oxide or silica gel.

14. The pharmaceutical product according to claim 13, wherein the silica gel is included in the packaging material in an amount of 2 to 5 g based on a 125 ml HDPE bottle.

15. A method of improving stability of a pharmaceutical preparation by reducing the formation of impurities comprising preparing the pharmaceutical preparation according to claim 1, the method comprising the steps of:
1) mixing a compound of Chemical Formula 1 or a pharmaceutically acceptable salt thereof with a pharmaceutically acceptable additive and then performing granulation to prepare a granule;
2) mixing the granule with a pharmaceutically acceptable additive and then adding a diluent to prepare a mixed granule, wherein the diluent is a mixture of mannitol and microcrystalline cellulose in a weight ratio of 0.50:1 to 3.2:1; and
3) formulating the mixed granules.

16. A method of treating cancer in a subject comprising administering a therapeutically effective amount of pharmaceutical preparation of claim 1, wherein the subject has been determined to have one or more EGFR or HER2 activating mutations.

17. The method of claim 16, wherein the subject has been determined to have one or more HER2 activating mutations selected from the group consisting of S310F/Y, I655V, V659E, R678Q, V697L, T733I, L755X, I767M, D769H/N/Y, V773M, V777L/M, L786V, V842I, and L869R.

18. The method of claim 16, wherein the subject has one or more EGFR activating mutations selected from the group consisting of EGFRvIII, R108K, R222C, A289T, P596L, G598V, Exon 20 insertion, E709K, G719X, V742I, E746_A750del, S768I, V769M, V774M, R831C, R831H, L858R, L861Q, and A864V.
